(19) United States
(12) Patent Application Publication
(10) Pub. No.: US 2003/0233713 A1
(43) Pub. Date: Dec. 25, 2003

(54) Kit for dyeing keratin fibers

(76) Inventors: Francis Xavier Quinn, Paris (FR); Pascal Giustiniani, Levallois Perret (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/411,328

(22) Filed: Apr. 11, 2003

Related U.S. Application Data
(60) Provisional application No. 60/375,428, filed on Apr. 26, 2002.

(30) Foreign Application Priority Data
Apr. 11, 2002 (FR) 02 04529

(51) Int. Cl. A61K 7/13

(52) U.S. Cl. 8/405; 8/406; 8/408

(57) ABSTRACT

A kit for dyeing keratin fibers, for example, human hair, comprising a basifying composition and an oxidizing composition comprising a molecular complex comprising at least one organic acid and at least one direct dye, as well as the oxidizing composition of the kit and the dyeing process using this kit.

This application claims benefit of U.S. Provisional Application No. 60/375,428, filed Apr. 26, 2002.

Disclosed herein is a kit for dyeing keratin fibers, for example, human hair, comprising a basifying composition and an oxidizing composition. Further disclosed herein are the oxidizing composition of the kit, the ready-to-use composition obtained from this kit and the dyeing process using this kit.

It is well-known practice to dye keratin fibers, for example, human hair, by oxidation dyeing with dye compositions comprising oxidation dye precursors, also known as oxidation bases, such as ortho- or para-phenylenediamines, ortho- or para-aminophenols and heterocyclic compounds. These oxidation bases may be colorless or weakly colored compounds, which, when combined with oxidizing products, can give rise to colored compounds by a process of oxidative condensation.

It is also well-known that the shades obtained with these oxidation bases may be varied by combining them with couplers or coloration modifiers. These couplers or coloration modifiers may be chosen, for example, from aromatic meta-diamines, meta-aminophenols, meta-diphenols and certain heterocyclic compounds.

The variety of molecules used as oxidation bases and couplers make it possible to obtain a wide range of colors.

The process of dyeing by oxidation dyeing, referred to as a “permanent” dyeing process, comprises applying to the keratin fibers a composition comprising oxidation bases or a mixture of oxidation bases and couplers, and an oxidizing agent, for example, hydrogen peroxide, leaving the composition to act and then rinsing the keratin fibers.

The colorations resulting from this process can be permanent, strong and fast with respect to external agents such as light, bad weather, washing, perspiration and rubbing. The process, which may be applied at basic pH, may produce a dyeing and a simultaneous lightening of the keratin fibers, which can be reflected in practice by the possibility of obtaining a final coloration that may be lighter than the original color. In addition, lightening of the keratin fibers may have an advantageous effect, for example, of generating a uniform color in the case of grey hair, and, in the case of naturally pigmented hair, of bringing out the color, i.e. of making it more visible.

It is also well-known practice to dye keratin fibers by direct dyeing. The process conventionally used in direct dyeing comprises applying to the keratin fibers direct dyes, which can be colored molecules and dyes that have an affinity for the keratin fibers, leaving these agents to act and then rinsing the keratin fibers. The standard direct dyes are, for example, dyes of the nitrobenzene, anthraquinone, nitropyridine, azo, methine, azomethine, xanthine, acridine, azine or triarylmethane dye type.

The colorations resulting from the direct dyeing can be particularly chromatic but may be temporary or semi-permanent due to the nature of the interactions which bind the direct dyes to the keratin fibers and their desorption from the surface and/or from the core of the keratin fibers. These factors can be responsible, for example, for their poor dyeing power and their poor wash-fastness or perspiration-fastness.

It is also well-known practice to use direct dyes in combination with oxidizing agents and alkaline agents to obtain lightening direct dyeing results.

For example, Patent Application No. FR 2 741 798 describes dye compositions comprising direct dyes comprising at least one quaternized nitrogen atom of the azo or azomethine type, said compositions intending to be mixed extemporaneously at basic pH with an oxidizing composition. These compositions may produce colorations with uniform, fast and bright glints.

It is also well-known practice in Patent Application Nos. EP 850 638, EP 852 135, EP 850 637 and EP 850 636, to use direct dyes in oxidation dyeing by combining a cationic direct dye with an oxidation base.

However, direct dyes may be relatively unstable in these types of compositions, such as in alkaline or oxidizing solutions, which may make it difficult to use them, for example, in lightening direct dyeing compositions based on aqueous hydrogen peroxide solution and, further, for example, with a basifying agent, or, even further, for example, in oxidation dye compositions in combination with precursors of the oxidation base or coupler type.

In such a medium, cationic direct dyes may have a tendency to degrade, which may, for example, limit the shelf life of the dye compositions and also may, further, for example, limit the dyeing capacity of these compositions.

Disclosed herein is a system for stabilizing cationic direct dyes intended to be used in oxidation dyeing or in lightening dyeing, that can, at the same time, maintain their affinity for the keratin fibers and also their coloring power.

Also disclosed herein is a kit for dyeing keratin fibers, comprising

a basifying composition comprising, in a medium suitable for dyeing, at least one alkaline agent, and

an oxidizing composition with a pH of less than or equal to 7, comprising, in a medium suitable for dyeing, at least one oxidizing agent and a molecular complex suspended in the medium, wherein the molecular complex is formed from at least one direct dye and at least one organic acid that is insoluble or sparingly soluble in the oxidizing composition, the pKa of which is higher than the pH of the oxidizing composition and lower than the pH of the mixture of the oxidizing composition and of the basifying composition, provided that the difference between the pKa of the at least one organic acid and the pH of the oxidizing composition is at least one unit and the difference between the pKa of the at least one organic acid and the pH of the mixture is at least one unit.

Also disclosed herein is an oxidizing composition as defined above, and also a ready-to-use composition comprising, a mixture of an oxidizing composition and a basifying composition as defined above.

The presence of at least one cationic direct dye in the form of a molecular complex may make it possible to avoid the degradation of the dye, since the acid may play a protective role with respect to the at least one oxidizing agent. A composition that may be stable on storage and the dyeing properties of which may not degrade over time, can be obtained.

At the time of use, the oxidizing and basifying compositions are mixed together to form a ready-to-use composition. This can result in a change in the pH, which can bring about the dissolution of the molecular complex and the release of the at least one cationic direct dye into the medium, the dye then being available for dyeing.

The kit disclosed herein may, for example, be used in oxidation dyeing or in lightening direct dyeing.

In the kit disclosed herein, the pH of the basifying composition may range, for example, from 7 to 12, and the pH of the oxidizing composition may be, for example, less than or equal to 5.

As used herein, the at least one organic acid that is insoluble or sparingly soluble in the oxidizing composition is an acid whose solubility in the oxidizing composition at 15° C. is less than 10 g/l, for example, less than 5 g/l and further, for example, 1 g/l.

The at least one insoluble organic acid forming the molecular complex may be chosen, for example, from chenodeoxycholic acid, benzoic acid, cinnamic acid, cholic acid and deoxycholic acid.

The at least one direct dye may be chosen, for example, from neutral, cationic and anionic direct dyes. For example, the at least one direct dye may be chosen from cationic direct dyes.

The cationic direct dyes may, for example, bear at least one quaternary ammonium group, which may be chosen, for example, from cationic aminoanthraquinones dyes, cationic monoazo and diazo dyes, cationic methine dyes, cationic azomethine dyes and cationic naphthoquinone dyes. The cationic direct dyes may further, for example, be chosen from azo (—N═N—), azomethine (—N═C—) and methine (—C═C—) dyes comprising at least one quaternary ammonium group.

According to one embodiment, the cationic direct dyes are chosen from compounds of formula (I) below:

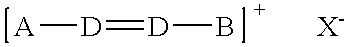

wherein

D, which may be identical or different, is chosen from a nitrogen atom and a CH radical;

A and B, which may be identical or different, are each chosen from aromatic groups that may form, optionally together with the D to which they are each attached, at lease one aromatic ring chosen from 4- to 6-membered aromatic rings or form at least two fused aromatic rings, wherein the aromatic rings may comprise at least one hetero atom and may be substituted with at least one entity chosen from halogen atoms and NR₁₁R₁₂ and OR₁₁ radicals, wherein R₁₁ and R₁₂, which may be identical or different, are each chosen from hydrogen, C₁-C₈ alkyl radicals, C₁-C₄ hydroxyalkyl radicals and a phenyl radical, and

X⁻ is an anion,

provided that at least one of the groups A and B comprises at least one cationic group, for example, at least one quaternary ammonium group.

The at least one cationic group may form an integral part of the aromatic group or may be borne by one of its substituents.

As used herein, the alkyl radical may be chosen from linear and branched, substituted and unsubstituted alkyl radicals. The aromatic groups may, for example, be chosen from phenyl, naphthyl, pyridine, pyrimidine, imidazole, pyrazole, piperazine, pyrrolidine, pyrrole, piperidine, and imidazolidine groups.

For example, the cationic direct dye may be chosen, for example, from

[8-[(p-aminophenyl)azol]-7-hydroxy-2-naphthyl]trimethylammonium chloride (also known as Basic Brown 16 or Arianor Mahogany 306002 in the Color Index),

3-[(4-amino-6-bromo-5,8-dihydro-1-hydroxy-8-imino-5-oxo-2-naphthyl)amino]-N,N,N-trimethylbenzeneaminium chloride (also known as Basic Blue 99 or Arianor Steel Blue 306004 in the Color Index),

7-hydroxy-8-[(2-methoxyphenyl)azo]-N,N,N-trimethyl-2-naphthaleneaminium chloride (also known as Basic Red 76 or Arianor Madder Red in the Color Index),

8-[(4-amino-2-nitrophenyl)azo]-7-hydroxy-2-naphthyl]trimethylammonium chloride (also known as Basic Brown 17 or Arianor Sienna Brown 306001 in the Color Index) and

3-[(4,5-dihydro-3-methyl-5-oxo-1-phenyl-1H-pyrazol-4-yl)azo]-N,N,N-trimethylbenzeneaminium chloride (also known as Basic Yellow 57 or Arianor Straw Yellow 306005 in the Color Index).

In one embodiment, the cationic direct dyes may be chosen from the compounds of formula (V) below:

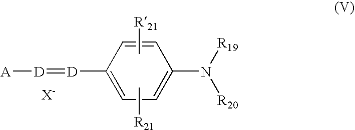

wherein:

D, which may be identical or different, is chosen from a nitrogen atom and a CH radical,

R₁₉ and R₂₀, which may be identical or different, are each chosen from a hydrogen atom; C₁-C₄ alkyl radicals which may be substituted with at least one radical chosen from —CN, —OH and —NH₂ radicals; and 4′-aminophenyl radicals; or form, together with a carbon atom of the benzene ring, an optionally oxygenated heterocycle which may be substituted with at least one radical chosen from C₁-C₄ alkyl radicals;

R₂, and R′₂₁, which may be identical or different, are each chosen from a hydrogen atom; halogen atoms chosen from chlorine, bromine, iodine and fluorine; cyano radicals; alkyl radicals; C₁-C₄ alkoxy radicals; and acetyloxy radicals;

X⁻ is an anion;

A is a group chosen from groups of formulae A₁ to A₁₉ below:

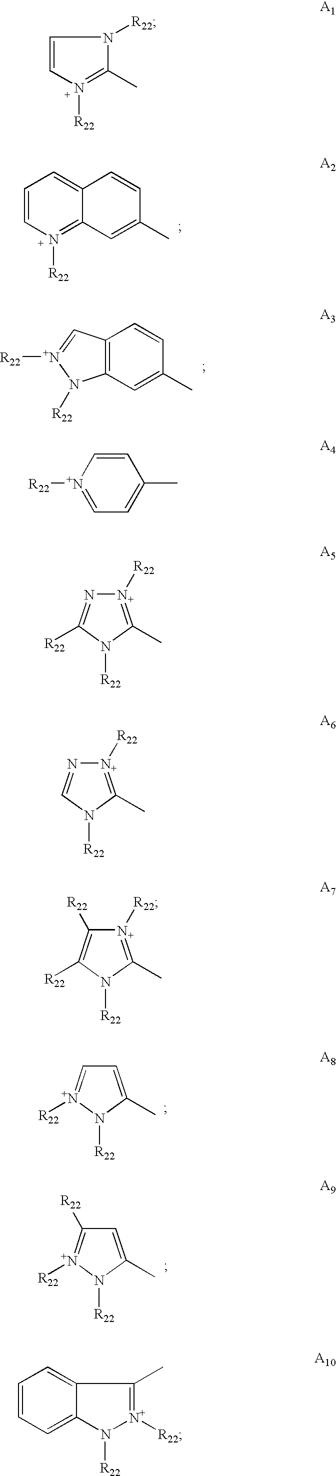

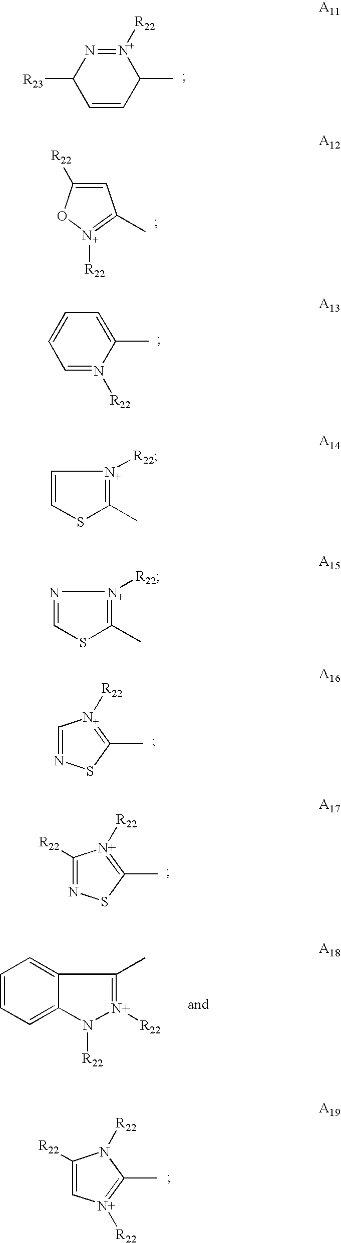

wherein:

R₂₂ is chosen from C₁-C₄ alkyl radicals which may be substituted with at least one hydroxyl radical, and R₂₃ is chosen from C₁-C₄ alkoxy radicals.

In one embodiment, A is A₁, R₁₉ and R₂₀, which may be identical or different, are each chosen from a hydrogen atom, C₁-C₄ alkyl radicals and 4′-aminophenyl radicals, and R₂₂ is chosen from C₁-C₄ alkyl radicals.

In another embodiment, the cationic direct dyes may be chosen, for example, from the compounds of formula (VI) below:

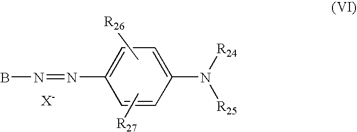

wherein:

R₂₄ is chosen from a hydrogen atom and C₁-C₄ alkyl radicals,

R₂₅ is chosen from a hydrogen atom; alkyl radicals which may be substituted with at least one entity chosen from a —CN radical and an amino group; and 4′-aminophenyl radicals, or forms together with R₂₄ an optionally oxygenated heterocycle which may be substituted with at least one radical chosen from C₁-C₄ alkyl radicals,

R₂₆ and R₂₇, which may be identical or different, are each chosen from a hydrogen atom; halogen atoms such as bromine, chlorine, iodine and fluorine; C₁-C₄ alkyl radicals; C₁-C₄ alkoxy radicals; and a —CN radical;

X⁻ is an anion;

B is a group chosen from the groups of formulae B₁ to B₆ below:

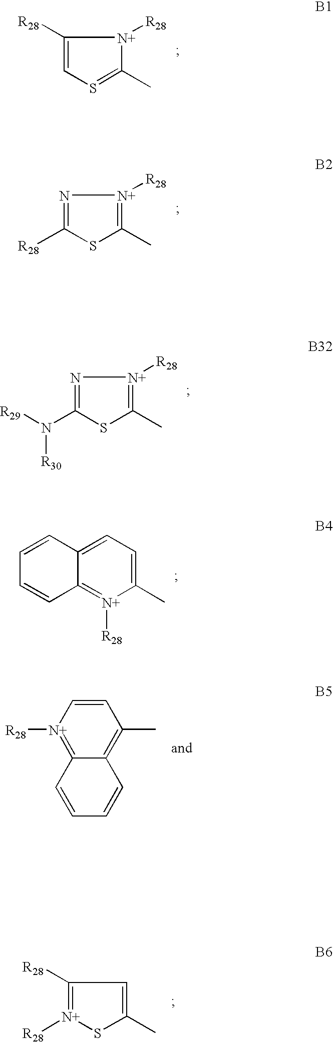

wherein R₂₈ is chosen from C₁-C₄ alkyl radicals, and R₂₉ and R₃₀, which may be identical or different, are each chosen from a hydrogen atom and C₁-C₄ alkyl radicals.

In another embodiment, the cationic direct dyes may be chosen, for example, from the compounds of formulae (VII) and (VII′) below:

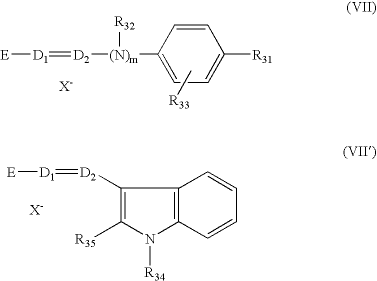

wherein:

R₃₁ is chosen from a hydrogen atom, C₁-C₄ alkoxy radicals, halogen atoms such as bromine, chlorine, iodine and fluorine, and amino radicals,

R₃₂ is chosen from a hydrogen atom and C₁-C₄ alkyl radicals or forms, together with a carbon atom of the benzene ring, an optionally oxygenated heterocycle which may be substituted with at least one group chosen from C₁-C₄ alkyl groups,

R₃₃ is chosen from a hydrogen atom and halogen atoms such as bromine, chlorine, iodine and fluorine,

R₃₄ and R₃₅, which may be identical or different, are each chosen from a hydrogen atom and C₁-C₄ alkyl radicals,

D₁ and D₂, which may be identical or different, are each chosen from a nitrogen atom and a —CH group,

m=0 or 1,

provided that when R₃₁ is an unsubstituted amino group, then D₁ and D₂ simultaneously represent a —CH group and m=0,

X⁻ is an anion,

E is a group chosen from the groups of formulae E₁ to E₈ below:

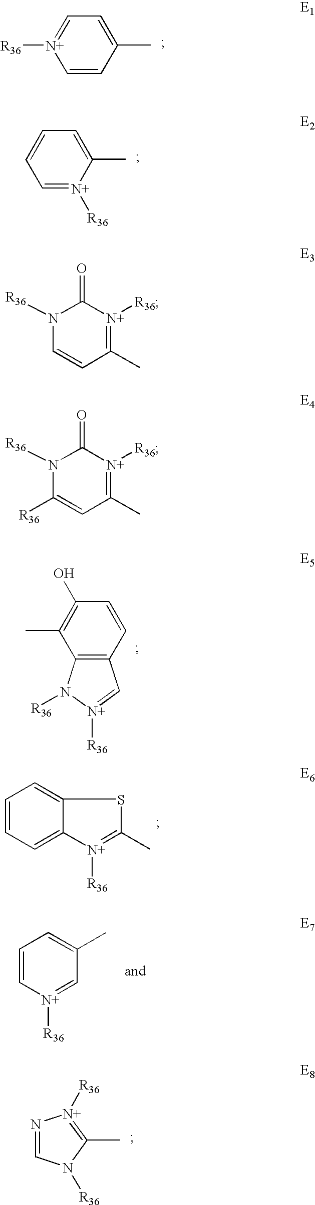

wherein R₃₆ is chosen from C₁-C₄ alkyl radicals;

when m=0 and D₁ is a nitrogen atom, then E may also be chosen from groups of formula E₉ below:

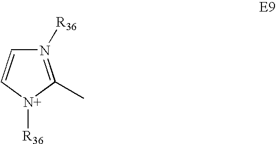

In one embodiment, compounds of formula (VII) may be used, wherein m=1, R₃₂ is chosen from a hydrogen atom and alkyl radicals and E is E₁.

The cationic direct dyes of formulae (V), (VI), (VII) and (VII′) that may be used herein are known compounds and are described, for example, in Patent Application Nos. WO 95/01772, WO 95/15144 and EP-A-0 714 954.

The cationic direct dye of formula (V) may, for example, be chosen from compounds of formulae (V₁) to (V₅₂) below:

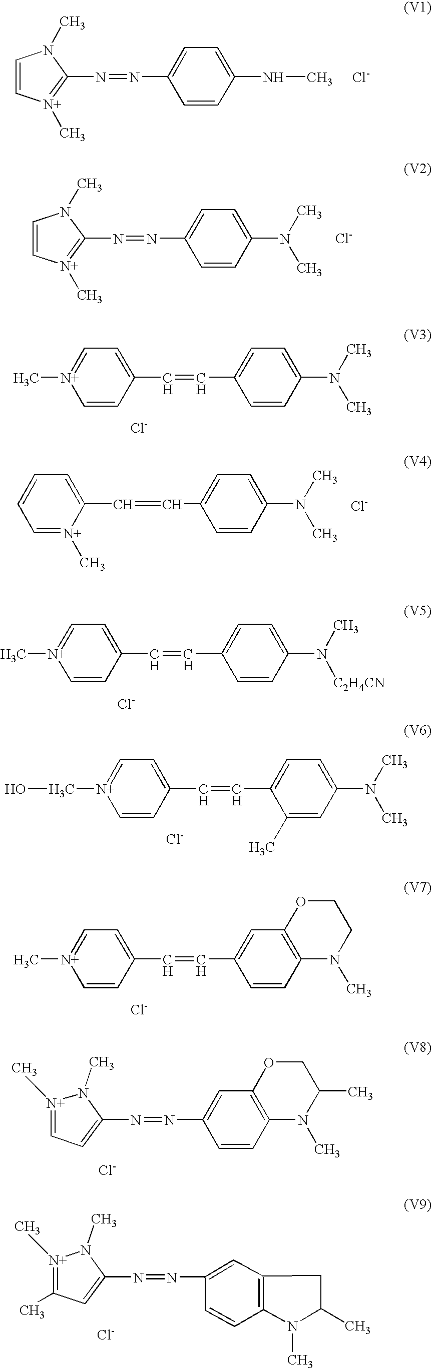

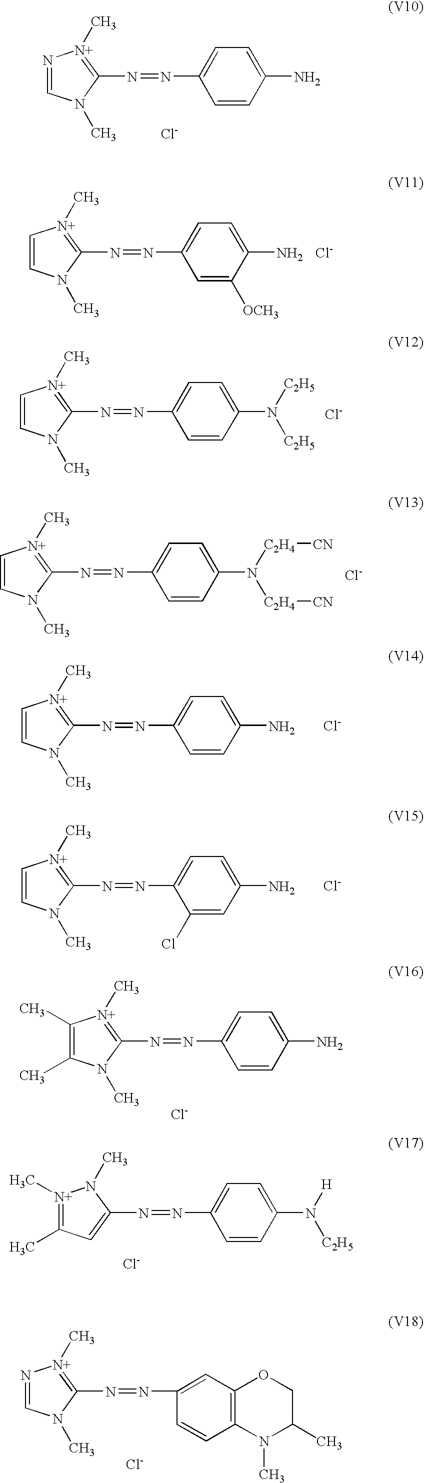

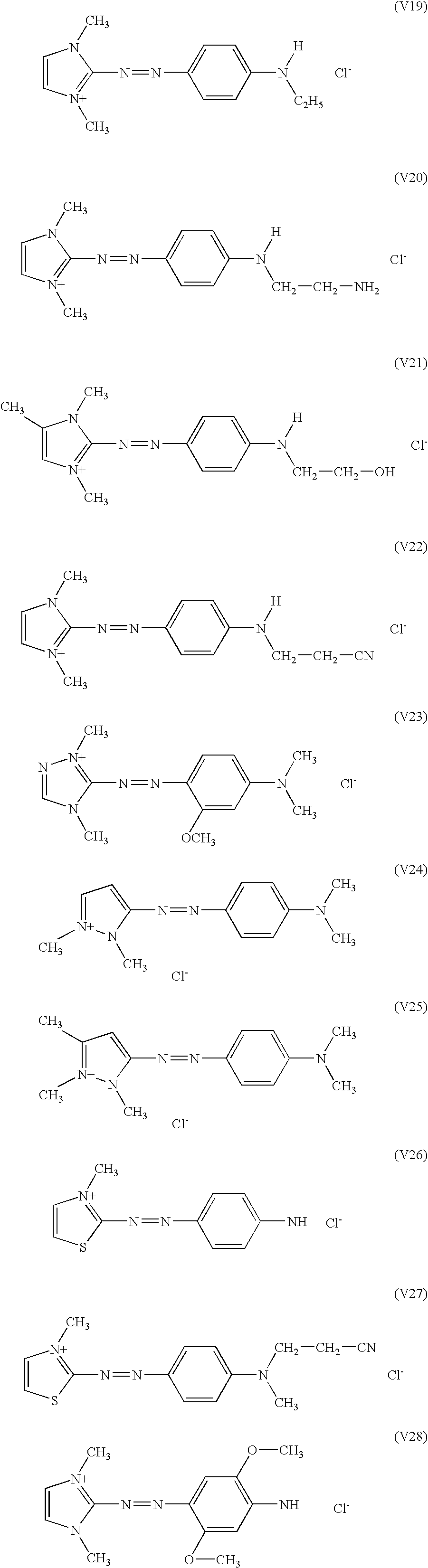

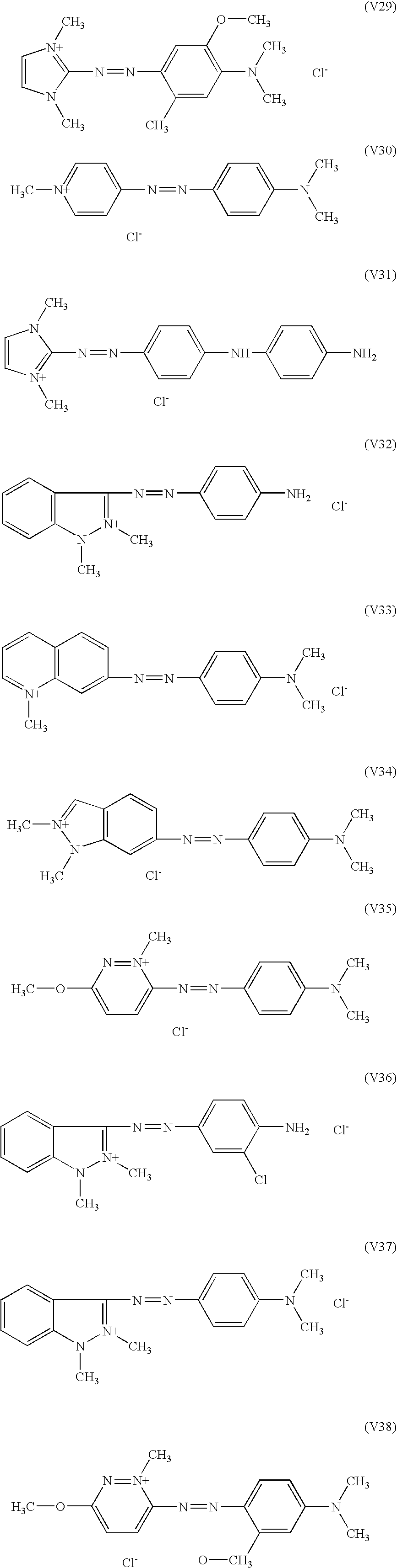

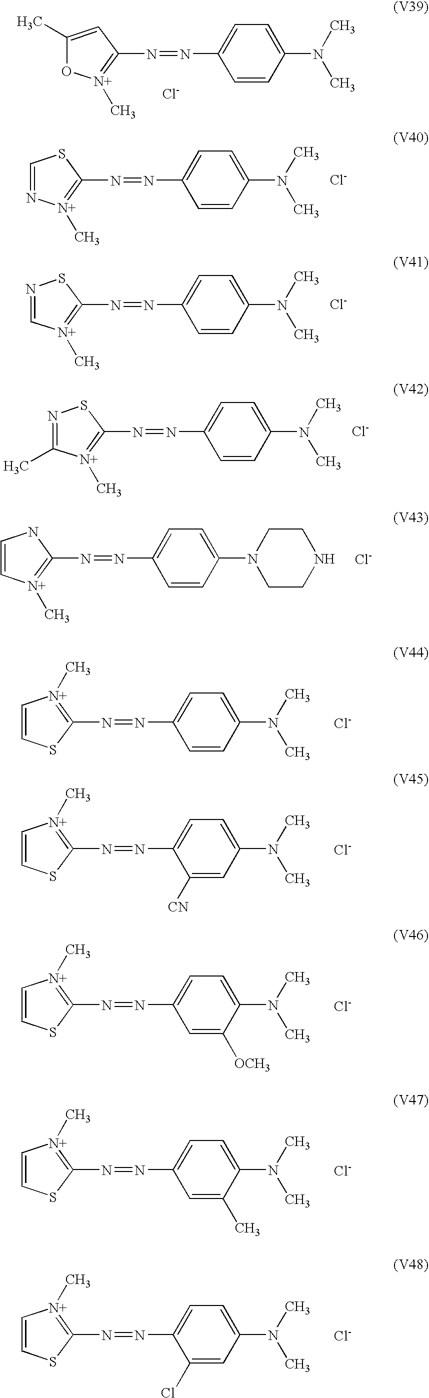

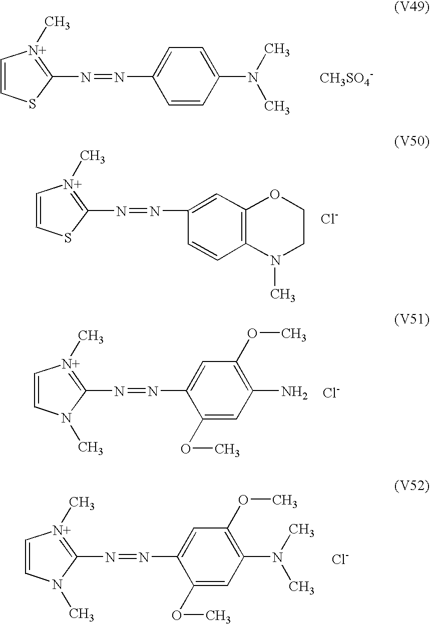

Among the compounds of formulae (V1) to (V52) described above, the compounds corresponding to formulae (V1), (V14) and (V31) may, for example, be used.

The cationic direct dyes of formula (VI), may, for example, be chosen from the compounds corresponding to formulae (VI1) to (VI12) below:

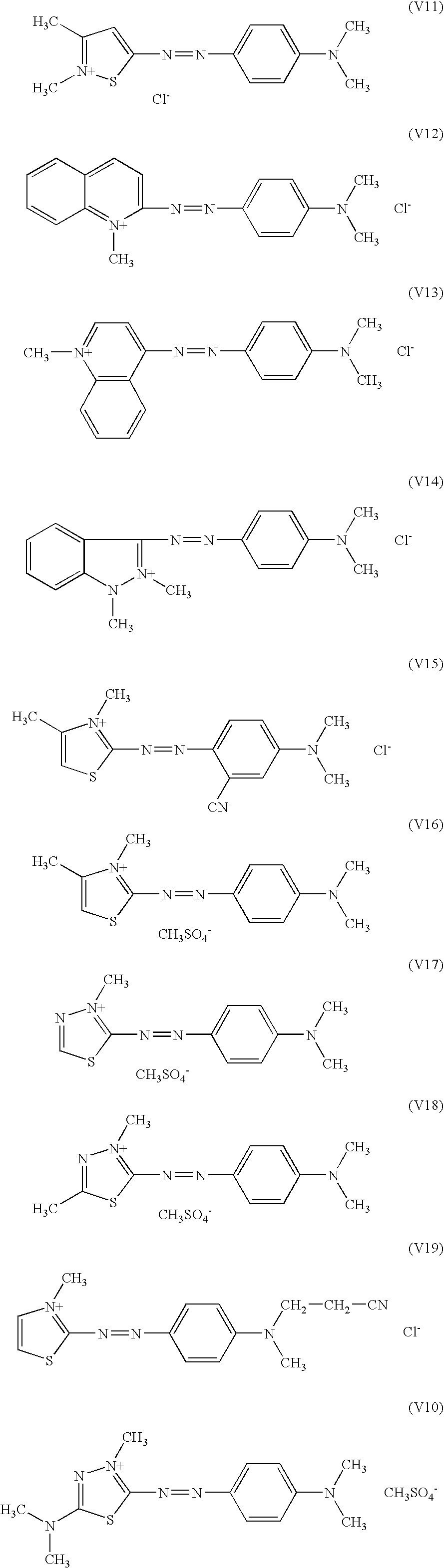

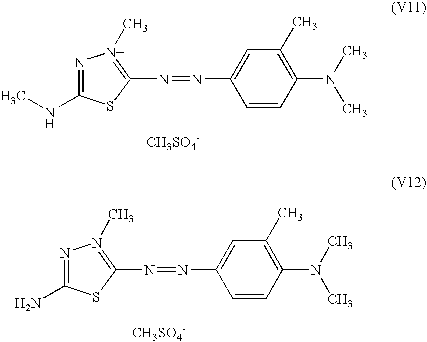

For example, the cationic direct dyes of formula (VII) may be chosen from compounds corresponding to formulae (VII1) to (VII18) below:

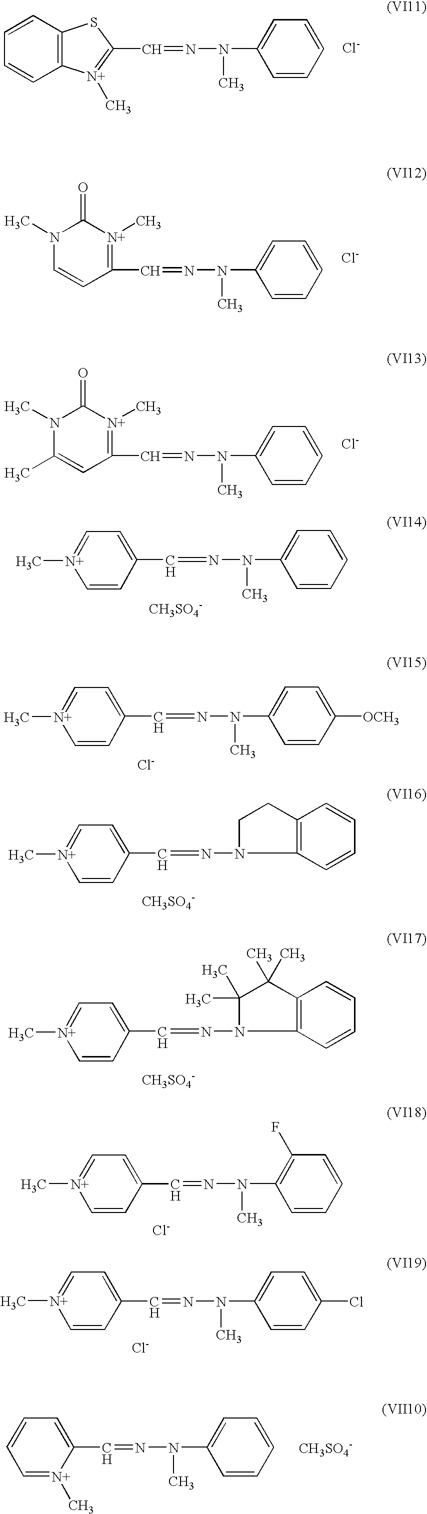

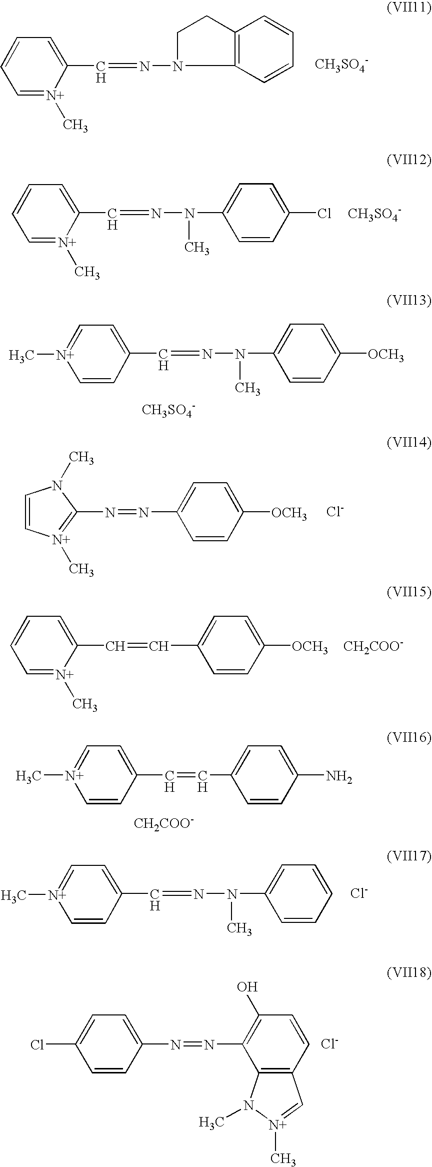

For example, among the compounds of formulae (VII1) to (VII18) described above, the compounds corresponding to formulae (VII4), (VII5) and (VII13) may be used.

The cationic direct dyes of formula (VII′) may, for example, be chosen from compounds corresponding to formulae (VII′1) to (VII′3) below:

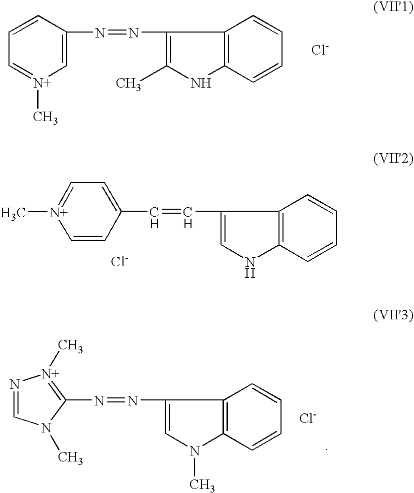

Other cationic direct dyes that may be used with the compositions disclosed herein may be chosen from those described in Patent Application Nos. FR 2 189 006, FR 2 285 851 and FR 2 140 205. For example, the cationic direct dyes may be chosen from the compounds of formulae (I)₁ to (I)₇₇ below:

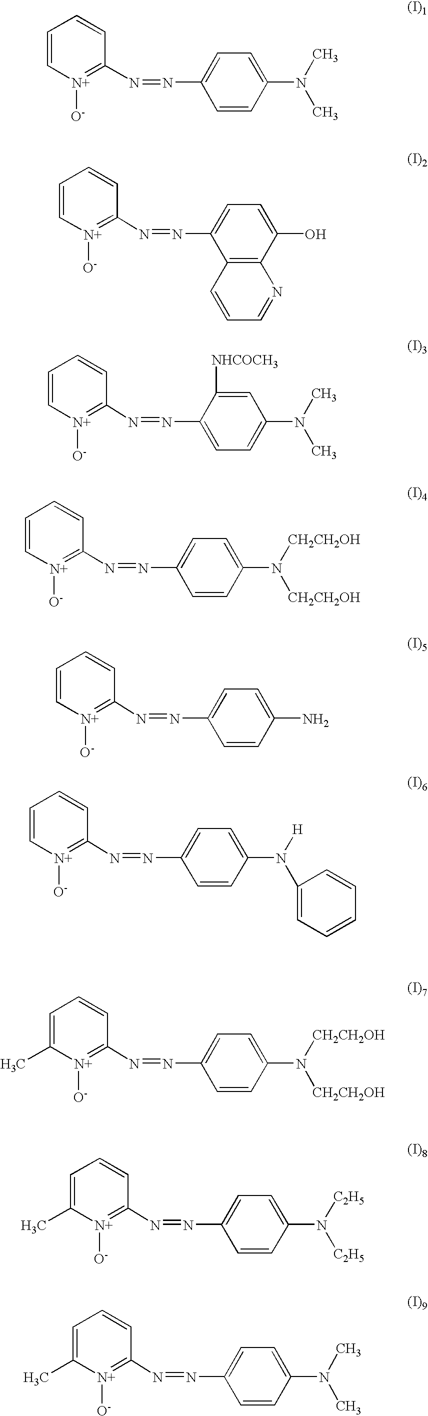

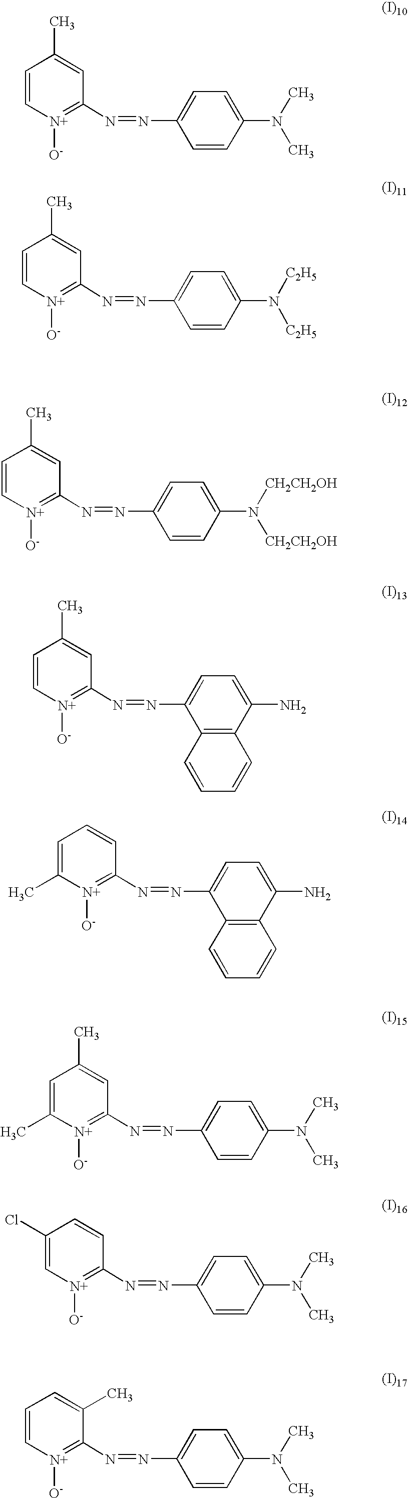

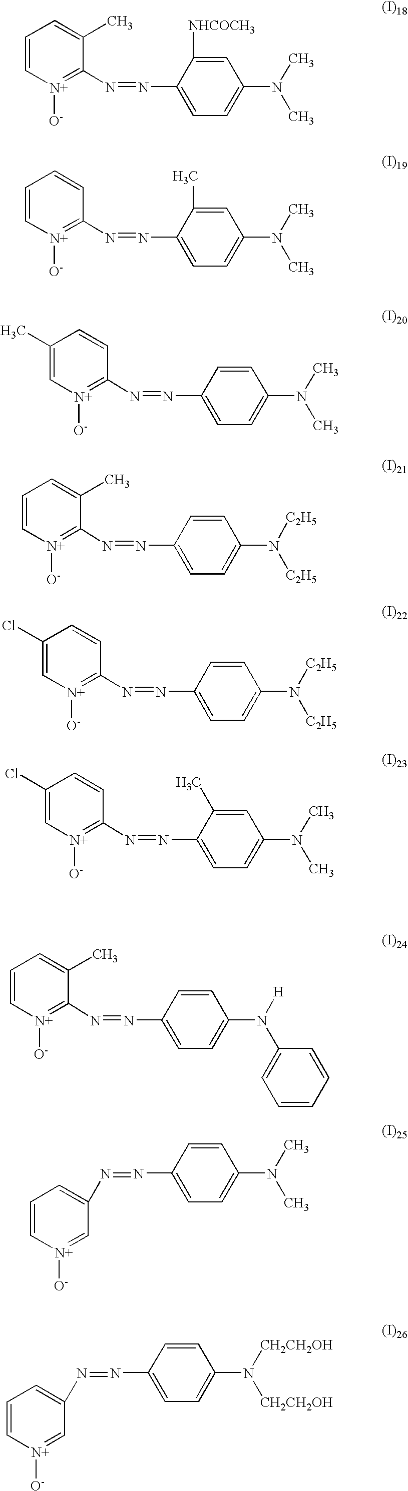

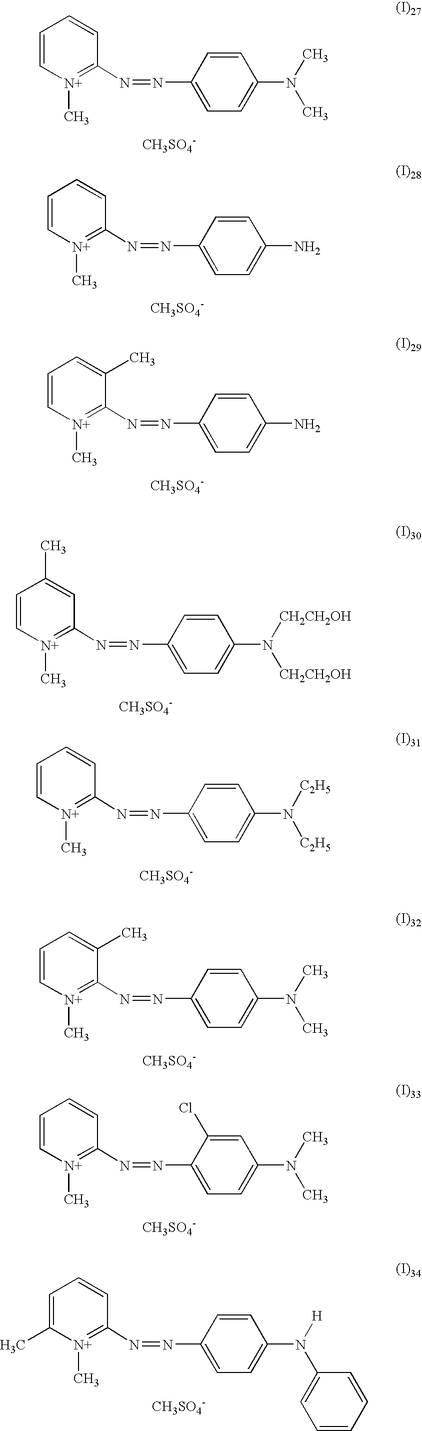

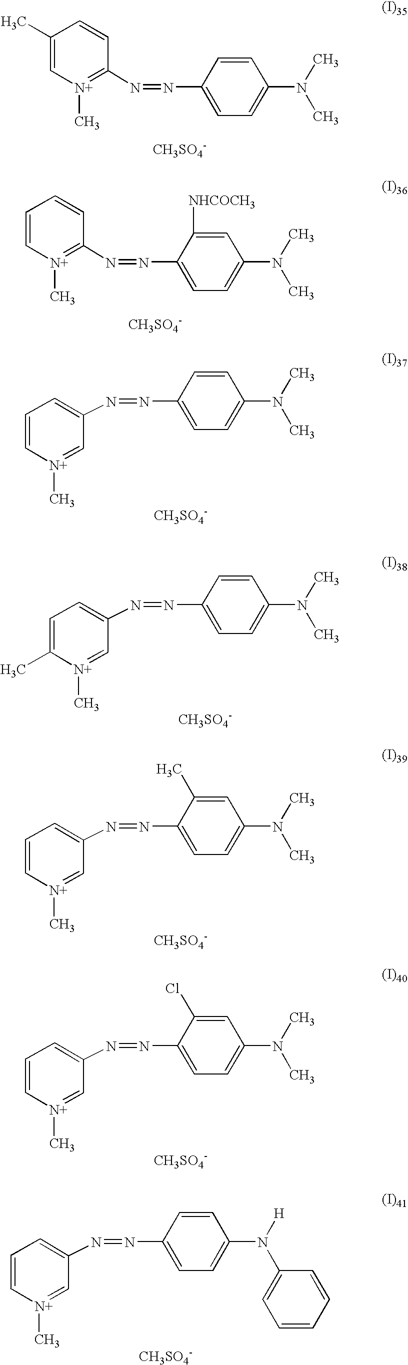

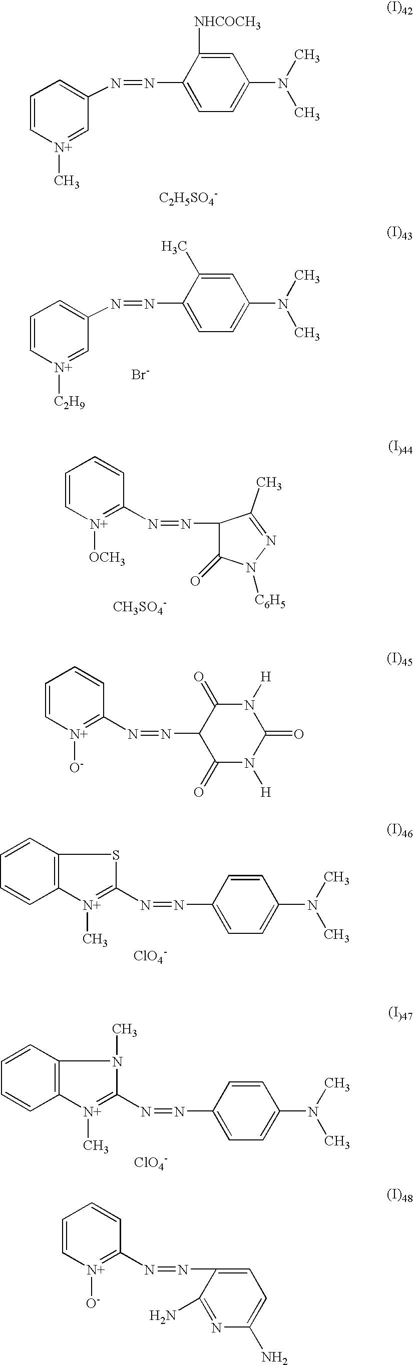

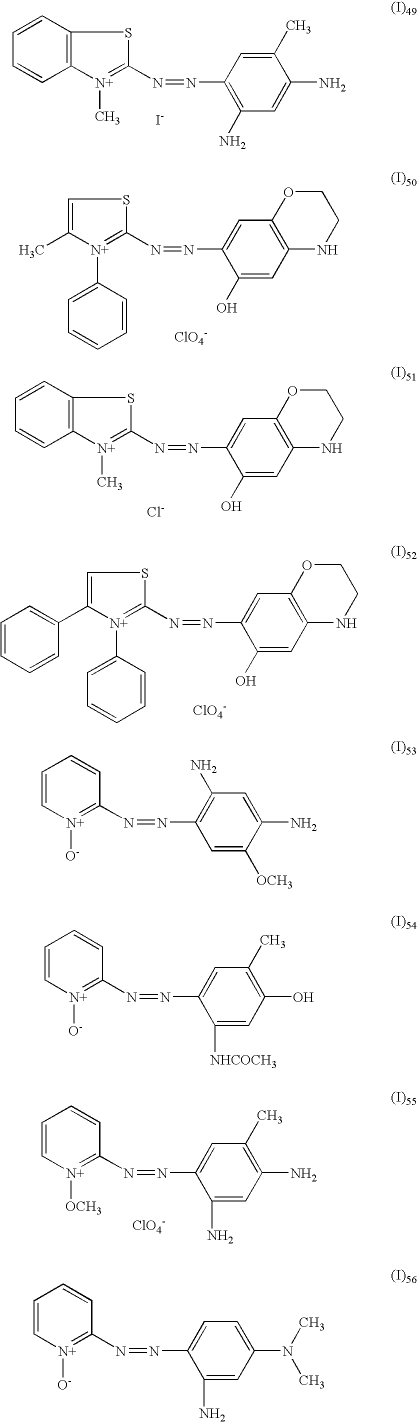

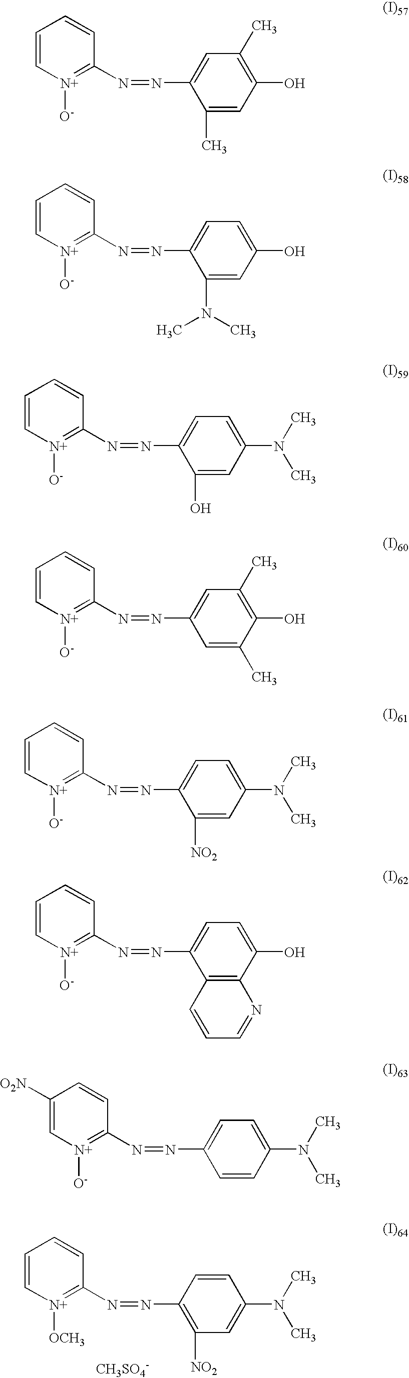

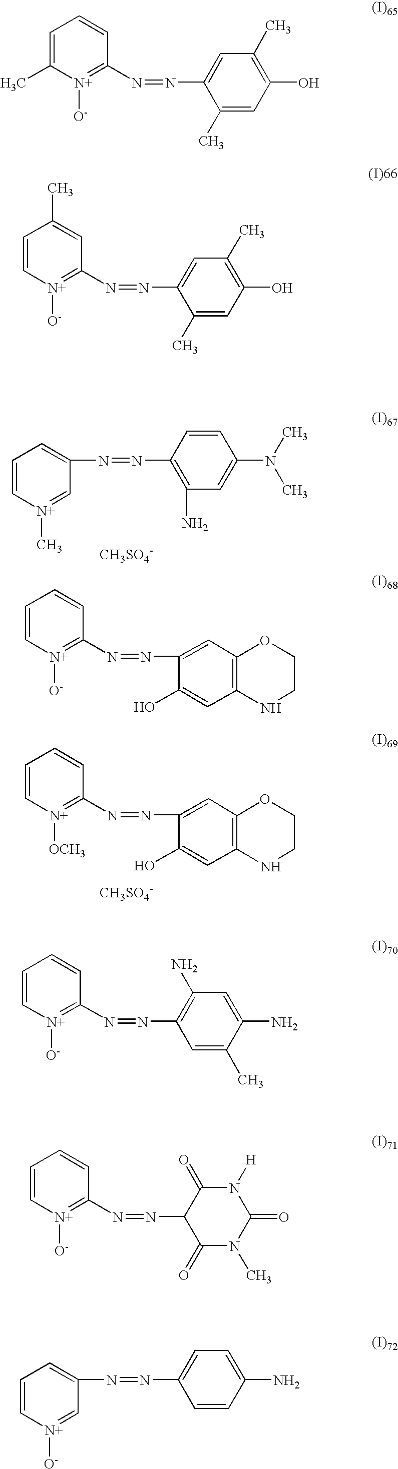

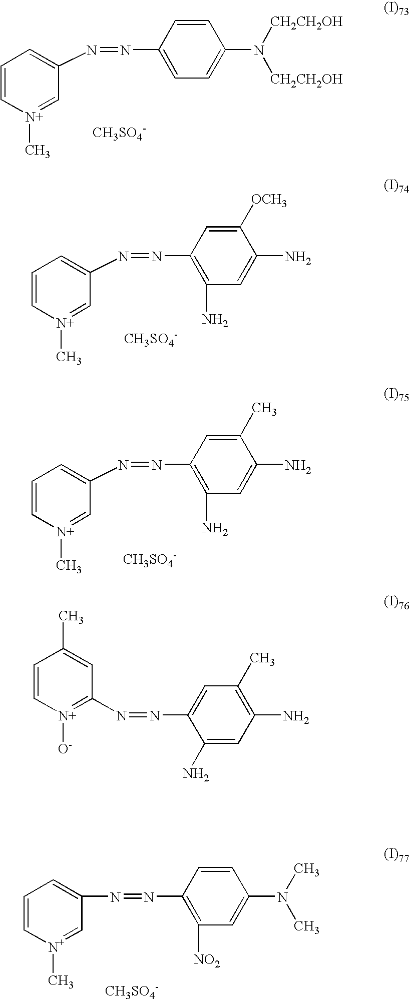

The molecular complex used herein may comprise, for example, an amount of the at least one organic acid and of the at least one direct dye such that the acid/dye mass ratio may be greater than 1/100, for example, greater than 1/30.

The amount of the molecular complex in the oxidizing composition may range, for example, from 0.5% to 50% by weight, further, for example, from 1% to 40% by weight, relative to the total weight of the oxidizing composition.

For example, according to one embodiment for preparing the molecular complex, the at least one organic acid and the cationic direct dye are dissolved in a suitable organic solvent, wherein the at least one organic acid complexes the cationic direct dye, thus forming a protection for the cationic direct dye. The solution thus obtained is poured into water with stirring in order to precipitate the molecular complex formed from the at least one organic acid and the cationic direct dye in solid form. After washing, filtering and drying, the molecular complex may be obtained in the form of a powder.

The oxidizing composition disclosed herein can be obtained by mixing the molecular complex with at least one oxidizing agent dissolved in a medium suitable for dyeing.

The at least one oxidizing agent may be any oxidizing agent that is conventional in the field of dyeing. For example, the at least one oxidizing agent may be chosen from hydrogen peroxide, urea peroxide, alkali metal bromates, persalts such as perborates and persulphates, and peracids.

In one embodiment, the basifying composition may comprise at least one alkaline agent. The at least one alkaline agent may be chosen, for example, from standard alkaline agents used in the field of dyeing. For example, the at least one alkaline agent may be chosen from aqueous ammonia, alkali metal carbonates, alkanolamines, sodium hydroxide, potassium hydroxide and the compounds of formula (III) below:

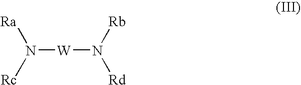

wherein W is chosen from propylene residues optionally substituted with at least one radical chosen from a hydroxyl group and C₁-C₄ alkyl radicals; Rₐ, R_{b}, R_{c}, and R_{d}, which may be identical or different, are each chosen from a hydrogen atom, C₁-C₄ alkyl and C₁-C₄ hydroxyalkyl radicals.

The basifying composition may further comprise at least one oxidation base chosen from those oxidation bases conventionally used in oxidation dyeing, for example para-phenylenediamines, bis(phenyl)alkylenediamines, para-aminophenols, ortho-aminophenols and heterocyclic bases, and the addition salts thereof.

The para-phenylenediamines may, for example, be chosen from compounds of formula (Ib) below, and the acid addition salts thereof:

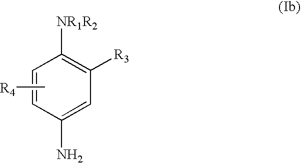

wherein:

R₁ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals and C₁-C₄ alkyl radicals substituted with at least one group chosen from nitrogen-comprising, phenyl and 4′-aminophenyl groups;

R₂ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals and C₁-C₄ alkyl radicals substituted with at least one nitrogen-comprising group;

R₁ and R₂ may also form, together with the nitrogen atom that bears them, a heterocycle,

R₃ is chosen from a hydrogen atom, halogen atoms such as chlorine, bromine, iodine and fluorine atoms, C₁-C₄ alkyl radicals, C₁-C₄ monohydroxyalkyl radicals, C₁-C₄ hydroxyalkoxy radicals, acetylamino(C₁-C₄)alkoxy radicals, mesylamino(C₁-C₄)alkoxy radicals and carbamoylamino(C₁-C₄)alkoxy radicals, and

R₄ is chosen from hydrogen and halogen atoms and C₁-C₄ alkyl radicals.

The nitrogen-comprising groups used herein may, for example, be chosen from amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

The para-phenylenediamines may, for example, be chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-N,N-bis(β-hydroxyethyl)-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β, β-dihydroxypropyl)-para-phenylenediamine, N-(4′-aminophenyl)-para-phenylenediamine, N-phenyl-para-phenylenediamine and 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine and 2-β-hydroxyethylamino-5-aminotoluene, and the acid addition salts thereof.

The para-phenylenediamines may, for example, be chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the acid addition salts thereof.

The double bases used in the compositions disclosed herein may, for example, be chosen from compounds corresponding to formula (IIb) below, and the acid addition salts thereof:

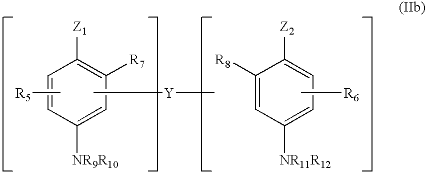

wherein:

Z₁ and Z₂, which may be identical or different, are each chosen from hydroxyl and —NH₂ radicals which may be substituted with at least one entity chosen from C₁-C₄ alkyl radicals and a linking arm Y;

the linking arm Y is chosen from linear and branched alkylene chains comprising from 1 to 14 carbon atoms, which may be interrupted by or terminated with at least one entity chosen from nitrogen-comprising groups and hetero atoms such as oxygen, sulphur and nitrogen atoms, and optionally substituted with at least one radical chosen from a hydroxyl radical and C₁-C₆ alkoxy radicals;

R₅ and R₆, which may be identical or different, are each chosen from hydrogen and halogen atoms, C₁-C₄ alkyl radicals, C₁-C₄ monohydroxyalkyl radicals, C₂-C₄ polyhydroxyalkyl radicals, C₁-C₄ aminoalkyl radicals and the linking arm Y; and

R₇, R₈, R₉, R₁₀, R₁₁ and R₁₂, which may be identical or different, are each chosen from a hydrogen atom, the linking arm Y and C₁-C₄ alkyl radicals;

provided that the compounds of formula (IIb) comprise only one linking arm Y per molecule.

The bis(phenyl)alkylenediamines may be chosen, for example, from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis-(ethyl)-N,N′-bis(4′-amino-3′-methylphenyl)ethylenediamine and 1,8-bis(2,5-diaminophenoxy)-3,6-dioxaoctane, and the acid addition salts thereof.

The para-aminophenols used in the compositions disclosed herein may be chosen, for example, from compounds corresponding to formula (IIIb) below, and the acid addition salts thereof:

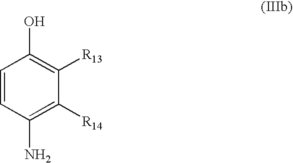

wherein:

R₁₃ is chosen from a hydrogen atom, halogen atoms, and C₁-C₄ alkyl, C₁-C₄ monohydroxyalkyl, (C₁-C₄)alkoxy(C₁-C₄)alkyl, C₁-C₄ aminoalkyl and hydroxy(C₁-C₄)alkylamino(C₁-C₄)alkyl radicals, and

R₁₄ is chosen from a hydrogen atom, halogen atoms, and C₁-C₄-alkyl, C₁-C₄ monohydroxyalkyl, C₂-C₄ polyhydroxyalkyl, C₁-C₄ aminoalkyl, C₁-C₄ cyanoalkyl and (C₁-C₄)alkoxy(C₁-C₄)alkyl radicals,

provided that at least one of the radicals R₁₃ and R₁₄ is a hydrogen atom.

The para-aminophenols may, for example, be chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the acid addition salts thereof.

The ortho-aminophenols may, for example, be chosen from 2-aminophenol, 2-amino-5-methylphenol, 2-amino-6-methylphenol and 5-acetamido-2-aminophenol, and the acid addition salts thereof.

The heterocyclic bases may, for example, be chosen from pyridine derivatives, pyrimidine derivatives and pyrazole derivatives.

The pyridine derivatives may, for example, be chosen from those described in Patent Nos. GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid addition salts thereof.

The pyrimidine derivatives may be chosen, for example, from those described in Patent Nos. DE 2 359 399, JP 88-169 571, JP 05-163 124, and EP 0 770 375 and Patent Application No. WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those described, for example, in Patent Application No. FR-A-2 750 048 such as pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine and 3-amino-5-methyl-7-imidazolylpropylamino-pyrazolo[1,5-a]pyrimidine and the acid addition salts thereof and the tautomeric forms thereof, where a tautomeric equilibrium exists.

The pyrazole derivatives, may be chosen, for example, from those compounds described in Patent Nos. DE 3 843 892 and DE 4 133 957 and Patent Application Nos. WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the acid addition salts thereof.

The at least one oxidation base may be present in an amount ranging, for example, from 0.001% to 10%, further, for example, from 0.005% to 6%, by weight, relative to the total weight of the basifying composition.

The basifying composition disclosed herein may further comprise at least one coupler chosen from those conventionally used for dyeing keratin fibers. The at least one coupler may be chosen, for example, from meta-phenylenediamines, meta-aminophenols, meta-diphenols, naphthalene-based couplers and heterocyclic couplers, and the addition salts thereof.

The at least one coupler may be chosen, for example, from 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, and 2,6-bis(β-hydroxyethylamino)toluene, and the addition salts thereof.

In one embodiment, the at least one coupler may, for example, be present in an amount ranging from 0.001% to 10% by weight, further, for example, from 0.005% to 6%, by weight, relative to the total weight of the basifying composition.

The addition salts that may be used herein for the at least one oxidation base and the at least one coupler disclosed herein may be chosen, for example, from the acid addition salts, such as hydrochlorides, hydrobromides, sulphates, citrates, succinates, tartrates, lactates, tosylates, benzenesulphonates, phosphates and acetates, and the base addition salts such as sodium hydroxide, potassium hydroxide, ammonia, amines and alkanolamines.

The medium suitable for dyeing, also known as the dye support, may be chosen from water and mixtures of water and at least one organic solvent to dissolve the compounds that may not be sufficiently water-soluble. The at least one organic solvent may be chosen, for example, from C₁-C₄ lower alkanols, such as ethanol and isopropanol; polyols and polyol ethers, such as 2-butoxyethanol, propylene glycol, propylene glycol monomethyl ether, diethylene glycol monoethyl ether and monomethyl ether, and aromatic alcohols, such as benzyl alcohol and phenoxyethanol.

The at least one organic solvent may be present in an amount ranging, for example, from 1% to 40% by weight, such as from 5% to 30% by weight, relative to the total weight of the dye composition.

The medium suitable for dyeing may also comprise at least one adjuvant chosen from adjuvants conventionally used in compositions for dyeing the hair, such as anionic, cationic, non-ionic, amphoteric and zwitterionic surfactants and mixtures thereof, anionic, cationic, non-ionic, amphoteric and zwitterionic polymers and mixtures thereof, mineral and organic thickeners such as anionic, cationic, non-ionic and amphoteric polymeric associative thickeners, antioxidants, penetrating agents, sequestering agents, fragrances, buffers, dispersants, conditioners such as volatile and non-volatile, modified and unmodified silicones, film-forming agents, ceramides, preserving agents and opacifiers.

The at least one adjuvant may be present in an amount ranging, for example, from 0.01% to 20% by weight, relative to the total weight of the composition.

Needless to say, a person skilled in the art will take care to select this or these optional additional compound(s) such that the advantageous properties intrinsically associated with the oxidation dye composition disclosed herein are not, or are not substantially, adversely affected by the envisaged addition(s).

Also disclosed herein is a process for dyeing keratin fibers, for example, human keratin fibers such as hair, using the kit disclosed herein.

This process comprises mixing the oxidizing composition and the basifying composition to form a ready-to-use composition. This ready-to-use composition may then be applied to the keratin fibers. After an action time ranging from 3 to 50 minutes, for example, from 5 to 30 minutes, the keratin fibers may be rinsed, washed with shampoo, rinsed again and then dried.

Before applying the ready-to-use composition to the keratin fibers, it may be necessary to modify the pH of the composition with at least one agent chosen from acidifying and basifying agents usually used in the dyeing of keratin fibers and as defined above. The pH of the ready-to-use composition may be adjusted to a pH value ranging from 3 to 12, for example, from 5 to 11.

Also disclosed herein is a multi-compartment device comprising a first compartment comprising the basifying composition defined above and a second compartment comprising the oxidizing composition defined above. This device may be equipped, for example, with a means for applying the desired mixture to the hair, such as the devices described in Patent No. FR-2 586 913.

The examples that follow serve to illustrate the embodiments disclosed herein without, however, being limiting in nature.

### EXAMPLES

### Example 1

### Preparation of the Molecular Complex

3 g of chenodeoxycholic acid and 50 mg of dye (V14) were introduced into a 50 ml flask. 10 g of absolute ethanol and 5 g of acetone were then added and the mixture was stirred for 30 minutes. The solution obtained was poured into 500 ml of water with stirring. A precipitate formed, which was recovered by filtration through a No. 3 sinter funnel. After washing the precipitate with water (500 ml) with stirring, filtering and drying, 2.4 g of solids were recovered.

### Preparation of the Oxidizing Composition

0.5 g of the solids obtained above were suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to a composition (A) comprising:

| | | |
|---|---|---|
| para-Tolylenediamine | 0.2 | g |
| Resorcinol | 0.1 | g |
| para-Aminophenol | 0.5 | g |
| 2-Methyl-5β-hydroxyethylaminophenol | 0.35 | g |
| 2-Methyl-5-aminophenol | 0.3 | g |
| Oleic acid | 2.7 | g |
| Monoethanolamine | 0.6 | g |
| Cetylstearyl alcohol | 16 | g |
| Oleocetyl alcohol containing 30 mol of EO | 3.5 | g |
| Sequestering agent | 0.8 | g |
| Aqueous ammonia comprising 20.5% NH₃ | 10.3 | g |
| Ammonium thiolactate | 4.8 | g |
| Fragrance | qs | |
| Demineralized water | qs 100 | g |

The powder dissolved immediately to give a colored composition in which the dye was then available to dye the hair.

### Example 2

### Preparation of the Molecular Complex

3 g of chenodeoxycholic acid and 50 mg of dye (V2) were introduced into a 50 ml flask. 10 g of absolute ethanol were then added and the mixture was stirred for 30 minutes. The solution obtained was poured into 500 ml of water with stirring. A precipitate formed, which was recovered by filtration through a No. 3 sinter funnel. After washing the precipitate in 500 ml of water with stirring, filtering and drying, 2.4 g of solids were recovered.

### Preparation of the Oxidizing Composition

0.5 g of the solids obtained above were suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 3

### Preparation of the Molecular Complex

3 g of chenodeoxycholic acid and 50 mg of dye (VII4) were introduced into a 50 ml flask. 10 g of absolute ethanol was then added and the mixture was stirred for 30 minutes. The solution obtained was poured into 500 ml of water with stirring. A precipitate formed, which was recovered by filtration through a No. 3 sinter funnel. After washing the precipitate with water (500 ml) with stirring, filtering and drying, 2.4 g of solids were recovered.

### Preparation of the Oxidizing Composition

0.5 g of the solids obtained above were suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 4

### Preparation of the Molecular Complex

10 mg of dye (V14) and 1 g of molten benzoic acid (125° C.) were placed in a beaker. The mixture was allowed to cool to room temperature (15 minutes). The addition of molten benzoic acid (125° C.) was repeated twice. 3 g of solids were recovered.

### Preparation of the Oxidizing Composition

0.5 g of the solids obtained above was suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 5

### Preparation of the Molecular Complex

3 g of chenodeoxycholic acid were placed in a 30 ml flask. The chenodeoxycholic acid was brought to the melting point (170° C.). 50 mg of dye (V2) were then added with stirring. Once the dye had all dissolved, the mixture was allowed to cool to room temperature (60 minutes). 3 g of solids were recovered.

### Preparation of the Oxidizing Composition

0.5 g of the solids obtained above were suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 6

### Preparation of the Molecular Complex

3 g of chenodeoxycholic acid and 50 mg of the following dye:

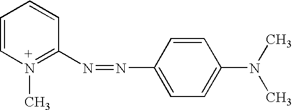

were placed in a 50 ml flask. 10 g of absolute ethanol was then added and the mixture was stirred for 30 minutes. The solution obtained was poured into 500 ml of water with stirring. A precipitate formed, which was recovered by filtration on a No. 3 sinter funnel. The precipitate was washed with 500 ml of water with stirring. After filtration, followed by drying, about 2.4 g of solids were recovered.

### Preparation of the Oxidizing Composition

0.5 g of the solids obtained above were suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 7

### Preparation of the Molecular Complex

3 g of chenodeoxycholic acid and 50 mg of the following dye:

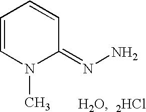

were placed in a 50 ml flask. 10 g of absolute ethanol was then added and the mixture was stirred for 30 minutes. The solution obtained was poured into 500 ml of water with stirring, at 50° C. A precipitate formed, which was recovered by filtration on a No. 3 sinter funnel. The precipitate was washed with 500 ml of water with stirring. After filtration, followed by drying, about 2.4 g of solids were recovered.

### Preparation of the Oxidizing Composition

0.5 g of the solids obtained above was suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 8

### Preparation of the Molecular Complex

3 g of chenodeoxycholic acid and 50 mg of the following dye:

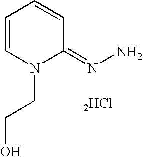

were placed in a 50 ml flask. 10 g of absolute ethanol was then added and the mixture was stirred for 30 minutes. The solution obtained was poured into 500 ml of water with stirring, at 50° C. A precipitate formed, which was recovered by filtration on a No. 3 sinter funnel. The precipitate was washed with 500 ml of water with stirring. After filtration, followed by drying, about 2.4 g of solids were recovered.

### Preparation of the Oxidizing Composition

0.5 g of the solids obtained above were suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

### Example 9

### Preparation of the Molecular Complex

3 g of chenodeoxycholic acid and 50 mg of the following dye:

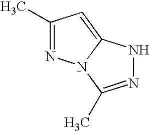

were placed in a 50 ml flask. 10 g of absolute ethanol were then added and the mixture was stirred for 30 minutes. The solution obtained was poured into 500 ml of water with stirring, at 50° C. A precipitate formed, which was recovered by filtration on a No. 3 sinter funnel. The precipitate was washed with 500 ml of water with stirring. After filtration, followed by drying, about 2.4 g of solids were recovered.

### Preparation of the Oxidizing Composition

0.5 g of the solids obtained above was suspended in 10 ml of 12-volumes H₂O₂. The powder was stable in the peroxide solution. It was insoluble in the solution and the color of the powder remained unchanged after storage for 2 months at 45° C.

### Preparation of the Ready-to-Use Composition

The suspension of powder in the peroxide was added to composition (A) described above. The powder dissolved immediately to give a colored composition in which the dye was then available to dye the hair.

What is claimed is:

1. A kit for dyeing keratin fibers, comprising
a basifying composition comprising, in a medium suitable for dyeing, at least one alkaline agent; and
an oxidizing composition with a pH of less than or equal to 7 comprising, in a medium suitable for dyeing, at least one oxidizing agent and a molecular complex suspended in the medium, wherein the molecular complex is formed from at least one direct dye and at least one organic acid that is insoluble or sparingly soluble in the oxidizing composition, the pKa of which is higher than the pH of the oxidizing composition and lower than the pH of the mixture of the oxidizing composition and of the basifying composition, provided that the difference between the pKa of the at least one organic acid and the pH of the oxidizing composition is at least one unit and the difference between the pKa of the at least one organic acid and the pH of the mixture is at least one unit.

2. The kit according to claim 1, wherein the pH of the basifying composition ranges from 7 to 12 and the pH of the oxidizing composition is less than or equal to 5.

3. The kit according to claim 1, wherein the at least one organic acid forming the molecular complex is chosen from chenodeoxycholic acid, benzoic acid, cinnamic acid, cholic acid and deoxycholic acid.

4. The kit according to claim 1, wherein the at least one direct dye is chosen from neutral, cationic, and anionic direct dyes.

5. The kit according to claim 4, wherein the at least one direct dye is chosen from cationic direct dyes.

6. The kit according to claim 5, wherein the cationic direct dyes bear at least one quaternary ammonium group.

7. The kit according to claim 6, wherein the cationic direct dyes are chosen from cationic aminoanthraquinone dyes, cationic monoazo dyes, cationic diazo dyes, cationic methine dyes, cationic azomethine dyes and cationic naphthoquinone dyes.

8. The kit according to claim 7, wherein the cationic direct dyes are chosen from azo dyes, azomethine dyes and methine dyes bearing at least one quaternary ammonium group.

9. The kit according to claim 5, wherein the cationic direct dyes are chosen from compounds of formula (I) below: 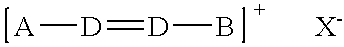
wherein:
Ds, which may be identical or different, are each chosen from a nitrogen atom and a CH radical,
A and B, which may be identical or different, are each chosen from aromatic groups that may form, optionally together with the D to which they are each attached, at least one aromatic ring chosen from 4- to 6-membered aromatic rings or form at least two fused aromatic rings, these aromatic rings possibly comprising at least one hetero atom and possibly being substituted with at least one entity chosen from halogen atoms and NR₁₁R₁₂ and OR₁₁ radicals, wherein R₁₁ and R₁₂, which may be identical or different, are each chosen from hydrogen, C₁-C₈ alkyl radicals, C₁-C₄ hydroxyalkyl radicals and a phenyl radical, and
X⁻ is an anion,
provided that at least one of the groups A and B comprises at least one cationic group.

10. The kit according to claim 9, wherein the at least one cationic group is a quaternary ammonium group.

11. The kit according to claim 5, wherein the cationic direct dyes are chosen from the compounds of formula (V) below: 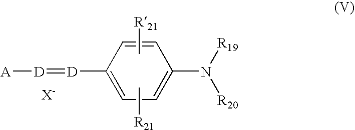
wherein:
Ds, which may be identical or different, are each chosen from a nitrogen atom and a CH radical,
R₁₉ and R₂₀, which may be identical or different, are each chosen from a hydrogen atom; C₁-C₄ alkyl radicals which may be substituted with at least one radical chosen from —CN, —OH and —NH₂ radicals; and 4′-aminophenyl radicals; or form, together with a carbon atom of the benzene ring, an optionally oxygenated heterocycle which may be substituted with at least one radical chosen from C₁-C₄ alkyl radicals;
R₂₁ and R′₂₁, which may be identical or different, are each chosen from a hydrogen atom; halogen atoms, cyano radicals, alkyl radicals, C₁-C₄ alkoxy radicals, and acetyloxy radicals.
X⁻ is an anion,
A is a group chosen from groups of formulae A₁ to A₁₉ below: 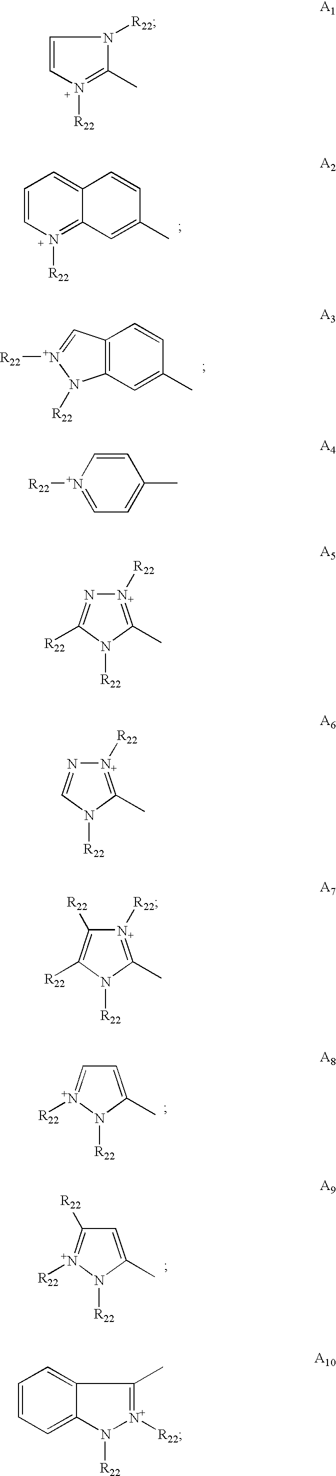 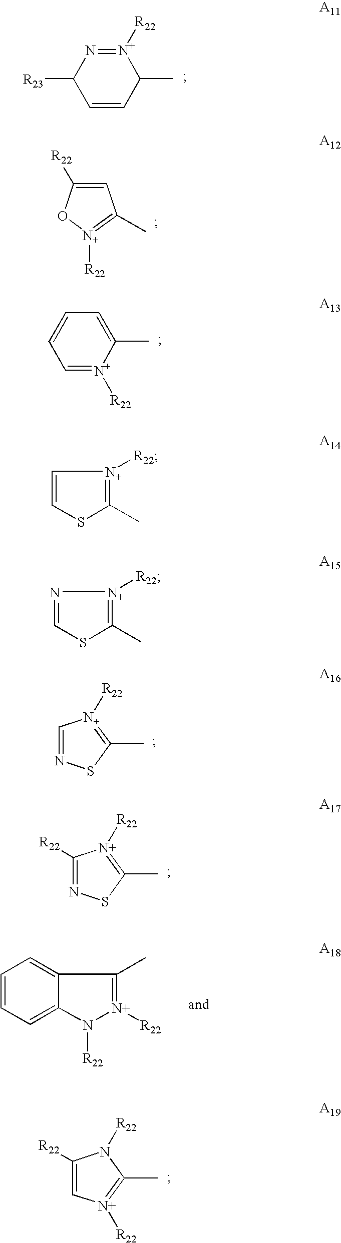
wherein:
R₂₂ is chosen from C₁-C₄ alkyl radicals which may be substituted with at least one hydroxyl radical, and
R₂₃ is chosen from C₁-C₄ alkoxy radicals.

12. The kit according to claim 11, wherein A is A1, R₁₉ and R₂₀, which may be identical or different, are each chosen from a hydrogen atom, C₁-C₄ alkyl radicals and 4′-aminophenyl radicals, and R₂₂ is chosen from C₁-C₄ alkyl radicals.

13. The kit according to claim 5, wherein the cationic direct dyes are chosen from the compounds of formula (VI) below: 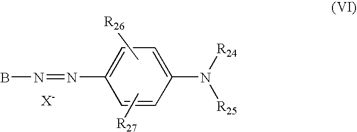
wherein:
R₂₄ is chosen from a hydrogen atom and C₁-C₄ alkyl radicals,
R₂₅ is chosen from a hydrogen atom; alkyl radicals which may be substituted with at least one entity chosen from a —CN radical and an amino group; and 4′-aminophenyl radicals, or forms with R₂₄ an optionally oxygenated heterocycle which may be substituted with at least one radical chosen from C₁-C₄ alkyl radicals,
R₂₆ and R₂₇, which may be identical or different, are each chosen from a hydrogen atom, halogen atoms, C₁-C₄ alkyl radicals, C₁-C₄ alkoxy radicals, and a —CN radical,
X⁻ is an anion,
B is a group chosen from groups of the formulae B₁ to B₆ below: 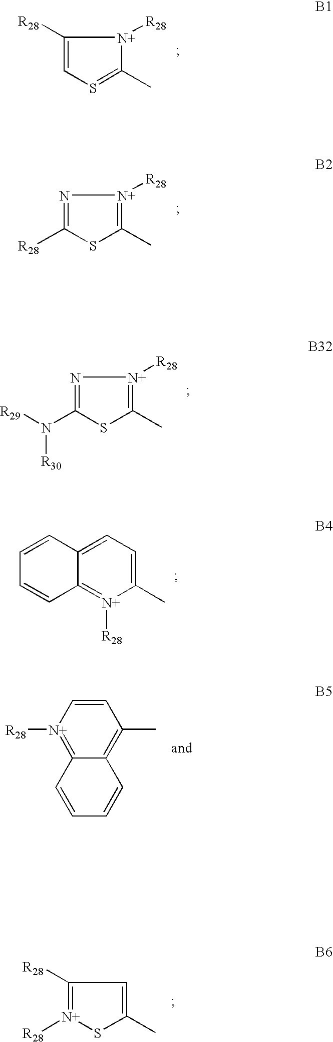
wherein R₂₈ is chosen from C₁-C₄ alkyl radicals, and R₂₉ and R₃₀, which may be identical or different, are each chosen from a hydrogen atom and C₁-C₄ alkyl radicals.

14. The kit according to claim 5, wherein the cationic direct dyes are chosen from the compounds of formulae (VII) and (VII′) below: 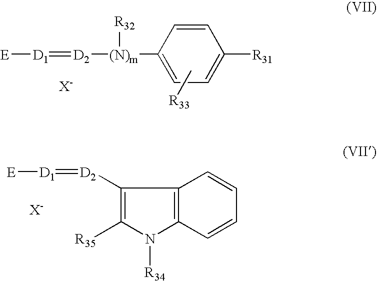
wherein:
R₃₁ is chosen from a hydrogen atom, C₁-C₄ alkoxy radicals, halogen atoms, and an amino radical,
R₃₂ is chosen from a hydrogen atom and C₁-C₄ alkyl radicals or forms, with a carbon atom of the benzene ring, an optionally oxygenated heterocycle which may be substituted with at least one group chosen from C₁-C₄ alkyl groups,
R₃₃ is chosen from a hydrogen atom and halogen atoms,
R₃₄ and R₃₅, which may be identical or different, are each chosen from a hydrogen atom and C₁-C₄ alkyl radicals,
D₁ and D₂, which may be identical or different, are each chosen from a nitrogen atom and a —CH group,
m=0 or 1,
provided that when R₃₁ is an unsubstituted amino group, then D₁ and D₂ simultaneously represent a —CH group and m=0,
X⁻ is an anion,
E is a group chosen from groups of formulae E₁ to E₈ below: 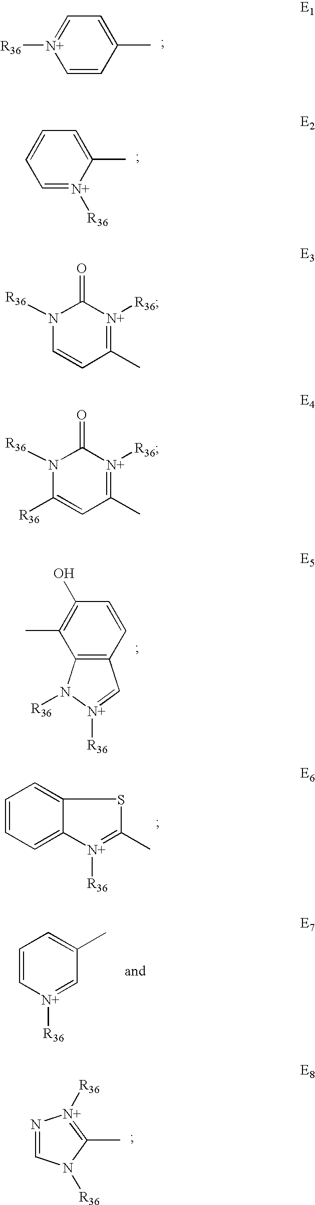
wherein R₃₆ is chosen from C₁-C₄ alkyl radicals;
when m=0 and D₁ is a nitrogen atom, then E may also be chosen from groups of formula E₉ below:

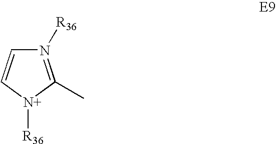

15. The kit according to claim 14, wherein the halogen atoms are chosen from bromine, chlorine, iodine, and fluorine.

16. The kit according to claim 14, wherein, in formula (VII), m=1, R₃₂ is chosen from a hydrogen atom and alkyl radicals, and E is E1.

17. The kit according to claim 5, wherein the cationic direct dyes are chosen from the dyes of the following formulae:

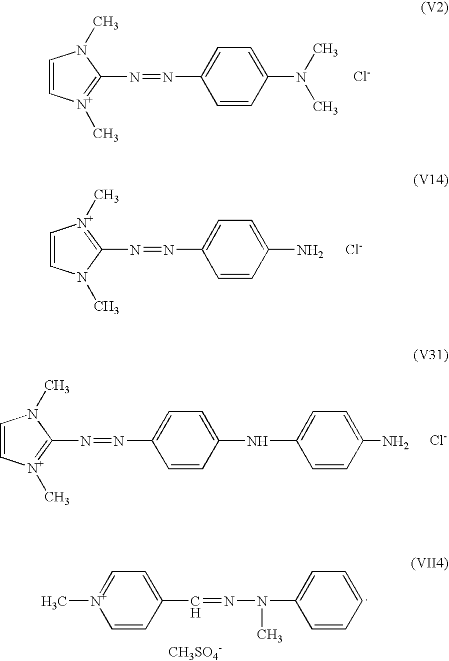

18. The kit according to claim 1, wherein the at least one organic acid and the at least one direct dye forming the molecular complex are present in an amount such that the acid/dye mass ratio is greater than 1/100.

19. The kit according to claim 18, wherein the at least one organic acid and the at least one direct dye forming the molecular complex are present in an amount such that the acid/dye mass ratio is greater than 1/30.

20. The kit according to claim 1, wherein the molecular complex is present in an amount ranging from 0.5% to 50% by weight, relative to the total weight of the oxidizing composition.

21. The kit according to claim 20, wherein the molecular complex is present in an amount ranging from 1% to 40% by weight, relative to the total weight of the oxidizing composition.

22. The kit according to claim 1, wherein the at least one oxidizing agent is chosen from hydrogen peroxide urea peroxide, alkali metal bromates, persalts and peracids.

23. The kit according to claim 1, wherein the at least one alkaline agent is chosen from aqueous ammonia, alkali metal carbonates, alkanolamines, sodium hydroxide, potassium hydroxide and the compounds of formula (III) below: 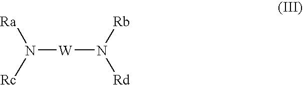
wherein W is chosen from propylene residues optionally substituted with at least one entity chosen from a hydroxyl group and C₁-C₄ alkyl radicals; Rₐ, R_{b}, R_{c} and R_{d}, which may be identical or different, are each chosen from a hydrogen atom and C₁-C₄ alkyl and C₁-C₄ hydroxyalkyl radicals.

24. The kit according to claim 1, wherein the basifying composition further comprises at least one oxidation base chosen from para-phenylenediamines, bis(phenyl)alkylenediamines, para-aminophenols, ortho-aminophenols and heterocyclic bases, and the addition salts thereof.

25. The kit according to claim 24, wherein the at least one oxidation base is present in an amount ranging from 0.001% to 10% by weight, relative to the total weight of the basifying composition.

26. The kit according to claim 25, wherein the at least one oxidation base is present in an amount ranging from 0.005% to 6% by weight, relative to the total weight of the basifying composition.

27. The kit according to claim 24, wherein the basifying composition further comprising at least one coupler chosen from meta-phenylenediamines, meta-aminophenols, meta-diphenols, naphthalene-based couplers and heterocyclic couplers, and the addition salts thereof.

28. The kit according to claim 27, wherein the at least one coupler is present in an amount ranging from 0.001% to 10% by weight, relative to the total weight of the basifying composition.

29. The kit according to claim 28, wherein the at least one coupler is present in an amount ranging from 0.005% to 6% by weight, relative to the total weight of the basifying composition.

30. An oxidizing composition, comprising, in a medium suitable for dyeing, at least one oxidizing agent and a molecular complex suspended in the medium, wherein the oxidizing composition has a pH of less than or equal to 7 and the molecular complex is formed from at least one direct dye and at least one organic acid that is insoluble or sparingly soluble in the oxidizing composition, the pKa of which is higher than the pH of the oxidizing composition and lower than the pH of the mixture of the oxidizing composition and of a basifying composition comprising in a medium suitable for dyeing, at least one alkaline agent, provided that the difference between the pKa of the at least one organic acid and the pH of the oxidizing composition is at least one unit and the difference between the pKa of the at least one organic acid and the pH of the mixture is at least one unit.

31. A process for the oxidation dyeing of keratin fibers, comprising
mixing an oxidizing composition and a basifying composition,
wherein the oxidizing composition has a pH of less than or equal to 7, and comprises, in a medium suitable for dyeing, at least one oxidizing agent and a molecular complex suspended in the medium,
wherein the molecular complex is formed from at least one direct dye and at least one organic acid that is insoluble or sparingly soluble in the oxidizing composition, the pKa of which is higher than the pH of the oxidizing composition and lower than the pH of the mixture of the oxidizing composition and of the basifying composition, provided that the difference between the pKa of the at least one organic acid and the pH of the oxidizing composition is at least one unit and the difference between the pKa of the at least one organic acid and the pH of the mixture is at least one unit; and
the basifying composition comprises, in a medium suitable for dyeing, at least one alkaline agent,
applying the mixture to the keratin fibers, and after an action time
rinsing the keratin fibers.

32. The process according to claim 31, wherein the keratin fibers are human keratin fibers.

33. The process according to claim 32, wherein the human keratin fibers are hair.

34. A multi-compartment device, comprising
a first compartment comprising a basifying composition comprising, in a medium suitable for dyeing, at least one alkaline agent; and
a second compartment comprising an oxidizing composition, with a pH of less than or equal to 7, comprising, in a medium suitable for dyeing, at least one oxidizing agent and a molecular complex suspended in the medium, wherein the molecular complex is formed from at least one direct dye and at least one organic acid that is insoluble or sparingly soluble in the oxidizing composition, the pKa of which is higher than the pH of the oxidizing composition and lower than the pH of the mixture of the oxidizing composition and of the basifying composition, provided that the difference between the pKa of the at least one organic acid and the pH of the oxidizing composition is at least one unit and the difference between the pKa of the at least one organic acid and the pH of the mixture is at least one unit.

35. A ready-to-use composition comprising a mixture of
a basifying composition comprising, in a medium suitable for dyeing, at least one alkaline agent, and
an oxidizing composition with a pH of less than or equal to 7, comprising, in a medium suitable for dyeing, at least one oxidizing agent and a molecular complex suspended in the medium, wherein the molecular complex is formed from at least one direct dye and at least one organic acid that is insoluble or sparingly soluble in the oxidizing composition, the pKa of which is higher than the pH of the oxidizing composition and lower than the pH of the mixture of the oxidizing composition and of the basifying composition, provided that the difference between the pKa of the at least one organic acid and the pH of the oxidizing composition is at least one unit and the difference between the pKa of the at least one organic acid and the pH of the mixture is at least one unit.

36. The kit according to claim 1, wherein the keratin fibers are human keratin fibers.

37. The kit according to claim 36, wherein the human keratin fibers are hair.
